(19) United States
(12) Patent Application Publication
(10) Pub. No.: US 2023/0317299 A1
(43) Pub. Date: Oct. 5, 2023

(54) SYSTEMS AND METHODS FOR ENFORCING CONTACT TRACING

(71) Applicant: SIGNIFY HOLDING B.V., EINDHOVEN (NL)

(72) Inventors: JIN YU, LEXINGTON, MA (US); PETER DEIXLER, ARLINGTON, MA (US); DAKSHA YADAV, BOSTON, MA (US); ABHISHEK MURTHY, ARLINGTON, MA (US)

(21) Appl. No.: 18/021,573

(22) PCT Filed: Aug. 9, 2021
(86) PCT No.: PCT/EP2021/072134

§ 371 Date: Feb. 16, 2023

Related U.S. Application Data
(60) Provisional application No. 63/067,004, filed on Aug. 18, 2020.

(30) Foreign Application Priority Data
Aug. 26, 2020 (EP) 20192840.5

(51) Int. Cl. G16H 50/80; G16H 40/67; H04M 1/72412

(52) U.S. Cl. CPC G16H 50/80; G16H 40/67; H04M 1/72412

(57) ABSTRACT

A method for enforcing contact tracing is provided including: associating, via a processor associated with a plurality of connected sensors, a first person with a first mobile device based in part on a first distance between the first person and the first mobile device; determining, via the processor, that the first person and the first associated mobile device are separated from each other by a second distance; notifying, via at least one light effect provided by an illumination device in communication with the processor, the first person that the first person and the first associated mobile device are separated from each other by the second distance. The method can further include: determining, via the processor, that a second person is positioned within predetermined proximity to the first person, and receiving, via the illumination device, contact tracing data on behalf of the first mobile device from the second mobile device.

### FIELD OF THE DISCLOSURE

The present disclosure is directed generally to a system and a method for enforcing contact tracing. More particularly, the present disclosure may preferably be directed to systems and methods for enforcing contact tracing using a lighting IoT system with embedded advanced sensor bundles (ASB) and supplementing mobile phone-based contact tracing information.

### BACKGROUND

Widespread social distancing has been adopted all around the world to fight the spread of COVID-19. To prevent the virus from spreading, social distancing requires people to isolate regardless of whether they have come into contact with the virus. Although social distancing has saved lives and eased the burden on hospitals, it comes at a high economic and social cost. Jobs have been lost, businesses have been closed, and a frozen economy has left many people anxious for restrictions to be eased and for life to get back to normal. However, until a vaccine is developed two key safety measures are necessary if social distancing restrictions are to be eased. The two key safety measures are widespread and easily accessible testing and contact tracing. Once widespread and easily accessible testing is in place, for those tests that come back positive, effective contact tracing must be in place to quickly identify people who have been in contact with those individuals who tested positive. Contact tracing is the process of contacting all people who have had contact with an individual who has tested positive for an infection or disease. Contact tracing has become a crucial element to blocking the spread of the recent and pandemic outbreak of COVID-19 by recording contacts.

Document WO2019/020477A1 discloses an infectious disease transmission tracking system based on a real-time locating system (RTLS).

Currently, mobile phone-based contact tracing applications use Bluetooth® low energy (BLE) technology to transmit anonymous “chirps” that may be received by other mobile phones nearby. The anonymous chirps are random, rotating numbers that do not reveal from where or to whom they were sent. If the user tests positive for an infection of disease, such as COVID-19, all phones that received the user's chirps are notified. Unfortunately, these contact tracing applications are only effective if individuals carry their phones at all times. However, this is not always the case. For example, individuals may intentionally or unintentionally leave their phones in their office or desk when they go to the restroom, make a quick photocopy, retrieve lunch or a snack, pick up a package, or have a quick conversation with their colleagues, etc. In these scenarios, the mobile phone-based contact tracing applications will have incomplete data and will not be able to effectively trace all of the person's contacts.

Additionally, contact tracing with BLE suffers from additional shortcomings. For instance, it is known that estimating people's proximity based on the received signal strength indicator (RSSI) of the BLE signals between the phones is compromised by walls, human bodies, pockets, phone orientation (landscape vs. portrait), or even proximity to several phones at once. In large open office spaces, these effects may lead to inaccurate detection of social proximity. Depending on the building material, a person in a different room may inadvertently be deemed to be in social distance.

Accordingly, there is an urgent need in the art for enforcing contact tracing and supplementing mobile phone-based contact tracing information to effectively trace all of a person's social contacts even when they are not carrying their mobile device such as a mobile phone.

### SUMMARY OF THE INVENTION

The present disclosure is directed to inventive systems and methods for enforcing contact tracing and complementing mobile phone-based contact tracing in commercial settings. The invention is set out in the appended set of independent and dependent claims.

Generally, embodiments of the present disclosure are directed to improved systems and methods for enforcing contact tracing and supplementing mobile phone-based contact tracing information using any internet of things (IoT) system. Applicant has recognized and appreciated that mobile phone-based contact tracing is insufficient in the office where people tend to forget their phones when performing a quick action, such as going to the bathroom, retrieving food, or having a quick conversation with a colleague. These activities are likely to lead to brief chance encounters with people who are from different departments of the same company, or even from different office locations. These brief close encounters with a person who is possibly infected by a virus will be missed. The absence of contact tracing data for these brief single-occasion encounters deteriorates the overall contact tracing effectiveness, especially if the people never meet again on the same day. Various embodiments and implementations herein are directed to systems and methods of associating phones with persons, detecting if any of the persons has forgotten his/her phone and alerting the person that he/she has forgotten his/her phone. Additional embodiments and implementations herein are directed to systems and methods of using an IoT system with embedded ASBs to track persons who have forgotten their mobile devices and others detected nearby, where the IoT system records as proxy the BLE information from the mobile phones of the detected people and transmits such information to the mobile phones of the detected people. The systems and methods described herein can advantageously provide automated reinforcement of corporate policies by generating notifications of violations of the same (e.g., when a user has left his/her phone behind) and supplement mobile phone-based contact tracing information systems where needed.

Generally, in one aspect, a method for enforcing contact tracing is provided. The method includes associating, via a processor associated with a plurality of connected sensors, a first person with a first mobile device based at least in part on a first distance between the first person and the first mobile device; determining, via the processor, that the first person and the first associated mobile device are separated from each other by a second distance; and notifying, via at least one light effect provided by a first illumination device in communication with the processor, the first person that the first person and the first associated mobile device are separated from each other by the second distance.

In embodiments, the method further includes: determining, via the processor, when the first person and the first associated mobile device are separated from each other by the second distance, that a second person having a second mobile device is positioned in close proximity to the first person; and receiving, via the first illumination device, contact tracing data on behalf of the first mobile device from the second mobile device. In embodiments, the step of determining that the second person is positioned in close proximity comprises: determining that the second person is positioned within a third distance from the first person; or determining that the second person is positioned within the third distance from the first person for at least a predetermined amount of time.

Thereby, said ‘close proximity’ may be phrased, throughout the application, as ‘within a predetermined proximity’. Said predetermined proximity may be characterized as within social distance.

Hence, alternatively phrased, the method includes, determining, via the processor, when the first person and the first associated mobile device are separated from each other by the second distance, that a second person having a second mobile device is positioned within a predetermined proximity to the first person; and receiving, via the first illumination device, contact tracing data on behalf of the first mobile device from the second mobile device. In embodiments, the step of determining that the second person is positioned within a predetermined proximity comprises: determining that the second person is positioned within a third distance from the first person; or determining that the second person is positioned within the third distance from the first person for at least a predetermined amount of time.

In embodiments, the method further includes the step of transmitting, via the first illumination device, contact tracing data on behalf of the first mobile device to the second mobile device. In embodiments, the method further includes the steps of transmitting, via the first illumination device, the received contact tracing data to a second illumination device that is positioned closer to the first mobile device than the first illumination device, and transmitting, by the second illumination device, the transmitted contact tracing data to the first mobile device.

In embodiments, the associating step comprises calculating a mapping probability between the first person with the first mobile device and comparing the mapped probability between the first person with the first mobile device with another mapping probability between the first person or the first mobile device with a second person or a second mobile device.

In embodiments, the method further includes the step of tracking the first person through a space by recording a thermal profile or a radio shadow outline of the first person.

In embodiments, the method further includes the steps of: (i) activating a first mode of operation when it is determined that the first person is carrying the first associated mobile device; and (ii) tracking the first person in the first mode of operation. In embodiments, the method further includes the steps of: (i) determining that the first person and the first associated mobile device are separated from each other by the second distance for a period of time that meets or exceeds a predetermined threshold; and (ii) initiating a second mode of operation comprising retrieving identifying information of the first person from the first mobile device at least in part in response to determining that the first person and the first associated mobile device are separated from each other by the second distance for the period of time.

Generally, in another aspect, a system for enforcing contact tracing is provided. The system includes at least one beacon receiver configured to identify position information for locating a first mobile device; at least one temperature-sensitive sensor associated with a plurality of connected sensors configured to identify position information of a first person; and a processor associated with the plurality of connected sensors, wherein the processor is configured to associate the first mobile device with the first person based at least in part on a first distance between the first person and the first mobile device, wherein the first distance is based on the position information identified by the at least one beacon receiver and the at least one temperature-sensitive sensor. The processor is further configured to determine that the first person and the first associated mobile device are separated from each other by a second distance. The system further includes an illumination device configured to provide at least one light effect to notify the first person that the first person and the first associated mobile device are separated from each other by the second distance.

In embodiments, the processor is further configured to determine, when the first person and the first associated mobile device are separated from each other by the second distance, that a second person having a second mobile device is positioned in close proximity (or, as mentioned, differently phrased: within a predetermined proximity) to the first person, and the first illumination device is further configured to receive contact tracing data on behalf of the first mobile device from the second mobile device.

In embodiments, the processor is further configured to track the first person through a space by recording a thermal profile or radio shadow outline for the first person, and the processor is further configured to track the first person according to a first mode of operation prior to determining that the first person and the first associated mobile device are separated from each other by the second distance.

In embodiments, the processor is further configured to track the first person through the space according to a second mode of operation after it is determined that the first person and the first associated mobile device are separated from each other by the second distance for a period of time that meets or exceeds a predetermined threshold, wherein in the second mode of operation, the plurality of connected sensors are configured to retrieve identifying information of the first person from the first associated mobile device.

In embodiments, the first illumination device is further configured to transmit contact tracing data on behalf of the first mobile device to the second mobile device, and the first illumination device is further configured to transmit the received contact tracing data to a second illumination device positioned closer to the first mobile device than the first illumination device, and the second illumination device is configured to transmit the received contact tracing data to the first mobile device.

Generally, in a further aspect, a computer program product embodied on at least one non-transitory computer-readable storage medium and configured so as when executed on one or more processors of a lighting system performs the following: associate a first mobile device with a first person based on position information identified for the first mobile device and the first person; track the first person throughout a space; determine when the first person and the first associated mobile device are separated from each other by a first distance; detect, after determining that the first person and the first associated mobile device are separated from each other by the first distance, that a second person having a second mobile device is positioned in close proximity (or, as mentioned, differently phrased: within a predetermined proximity) to the first person; cause an illumination device to provide at least one light effect to notify the first person that the first person and the first associated mobile device are separated from each other by the first distance; and cause transmission of contact tracing data between the first and second mobile devices.

In various implementations, the processor described herein may take any suitable form, such as, one or more processors or microcontrollers, circuitry, one or more controllers, a field programmable gate array (FGPA), or an application-specific integrated circuit (ASIC) configured to execute software instructions. Memory associated with the processor may take any suitable form or forms, including a volatile memory, such as random-access memory (RAM), static random-access memory (SRAM), or dynamic random-access memory (DRAM), or non-volatile memory such as read only memory (ROM), flash memory, a hard disk drive (HDD), a solid-state drive (SSD), or other non-transitory machine-readable storage media. The term “non-transitory” means excluding transitory signals but does not further limit the forms of possible storage. In some implementations, the storage media may be encoded with one or more programs that, when executed on one or more processors and/or controllers, perform at least some of the functions discussed herein. It will be apparent that, in embodiments where the processor implements one or more of the functions described herein in hardware, the software described as corresponding to such functionality in other embodiments may be omitted. Various storage media may be fixed within a processor or may be transportable, such that the one or more programs stored thereon can be loaded into the processor so as to implement various aspects as discussed herein. Data and software, such as the algorithms or software necessary to analyze the data collected by the sensors, an operating system, firmware, or other application, may be installed in the memory.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the present disclosure.

FIG. 1A is an example schematic depiction of a lighting IoT system for contact tracing according to aspects of the present disclosure;

FIG. 1B is an example schematic depiction of a lighting IoT system for contact tracing according to aspects of the present disclosure;

FIG. 2 shows example processes for enforcing contact tracing and complementing mobile phone-based contact tracing according to aspects of the present disclosure;

FIG. 3 is an example depiction of associating mobile devices with people according to aspects of the present disclosure; and

FIG. 4 shows an example process for associating mobile devices with people according to aspects of the present disclosure.

### DETAILED DESCRIPTION OF EMBODIMENTS

The present disclosure describes various embodiments of systems and methods for enforcing contact tracing and supplementing contact tracing information in mobile phone-based contact tracing systems. Applicant has recognized and appreciated that it would be beneficial to provide any internet of things (IoT) system with the capability to notify a user when he/she has forgotten his/her mobile phone in commercial settings. Applicant has also recognized and appreciated that it would be beneficial to use the IoT system to track the user and the user's social contacts when he/she has forgotten his/her mobile phone. The IoT system can record as proxy information about the user's social contacts and provide such information to the user's mobile phone to supplement the mobile phone-based contact tracing information. The IoT system can also transmit as proxy information to the user's social contacts. Thus, the IoT system provides complementary information to mobile devices, the complementary information including data regarding social encounters which would have otherwise been missed by device-device contact tracing. The lighting system data can also be used to increase the confidence on whether two individuals who are carrying mobile devices are within social distance of each other. Exemplary goals of utilization of certain embodiments of the present disclosure are to use a lighting IoT system with embedded advanced sensor bundles (ASBs) to fill in for the shortcomings of contact tracing systems based on BLE only.

The present disclosure describes various embodiments of systems and methods for enforcing contact tracing using suitable illumination devices that are connected or connectable and sensor enabled such as ceiling recessed or surface mounted luminaires, suspended luminaires, wall mounted luminaires, and free floor standing luminaires, etc. Such existing infrastructures can be used as a backbone for the enforcement and complementary contact tracing functionalities described herein. Signify's SlimBlend® suspended luminaire is one example of a suitable illumination device equipped with integrated IoT sensors such as BLE beacon receivers and thermopile infrared sensors as described herein. In embodiments, the illumination device includes USB type connector slots for the receivers and sensors etc. Illumination devices including sensor ready interfaces are particularly well suited and already provide powering, digital addressable lighting interface (DALI) connectivity to the luminaire's functionality and a standardized slot geometry. Suspended luminaires or free floor standing luminaires including thermopile infrared sensors are advantageous because the sensors are arranged closer to humans and can detect higher temperatures of people. Additionally, the resolution of the thermopile sensor can be lower than for thermopile sensors mounted within a ceiling recessed or surface mounted luminaire mounted at approximately 3 m ceiling height.

The term “luminaire” as used herein refers to an apparatus including one or more light sources of same or different types. A given luminaire may have any one of a variety of mounting arrangements for the light source(s), enclosure/housing arrangements and shapes, and/or electrical and mechanical connection configurations. Additionally, a given luminaire optionally may be associated with (e.g., include, be coupled to and/or packaged together with) various other components (e.g., control circuitry) relating to the operation of the light source(s). Also, it should be understood that light sources may be configured for a variety of applications, including, but not limited to, indication, display, and/or illumination.

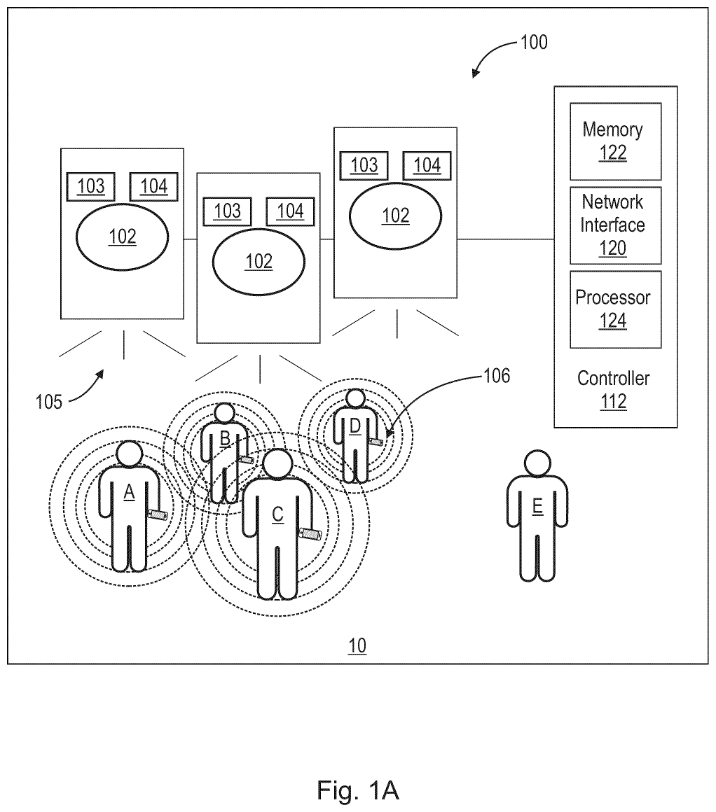

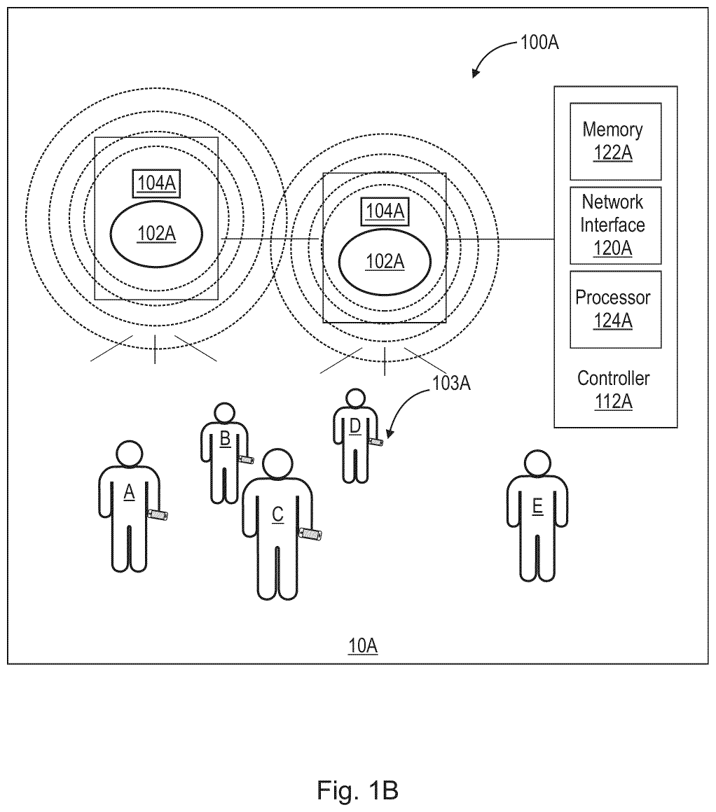

Referring to FIG. 1A, a schematic depiction of a lighting IoT system 100 for enforcing contact tracing in a space 10 is provided. The lighting IoT system 100 includes one or more overhead connected lighting networks that are equipped with a plurality of connected sensors (e.g., advanced sensor bundles (ASBs)). The overhead connected lighting networks refer to any interconnection of two or more devices (including controllers or processors) that facilitates the transmission of information (e.g., for device control, data storage, data exchange, etc.) between the two or more devices coupled to the network. Any suitable network for interconnecting two or more devices is contemplated including any suitable topology and any suitable communication protocols. The BLE and other sensor capabilities of the ASBs are used to accurately trace people within a building space. It should be appreciated that the lighting IoT system 100 can be configured in a typical office setting, a hotel, or any suitable alternative. The lighting IoT system 100 includes illumination devices 102 that may include one or more light-emitting diodes (LEDs). The LEDs may be active (i.e., turned on); inactive (i.e., turned off); or dimmed by a factor d, where 0≤d≤1. The value d=0 means that the LED is turned off whereas d=1 represents an LED that is at its maximum illumination. The illumination devices 102 may be arranged in a symmetric grid or, e.g., in a linear, rectangular, triangular or circular pattern. Alternatively, the illumination devices 102 may be arranged in any irregular geometry. It should be appreciated that the overhead connected lighting networks include the illumination devices 102, beacon receivers 103, and ASBs 104 ideally positioned to provide a sufficiently dense sensor network to cover a whole building indoor space. The beacon receivers 103 can be separate from the ASBs 104 or the beacon receivers 103 can be integrated within the ASBs 104. In embodiments where the beacon receivers 103 are separate from the ASBs, the beacon receivers 103 are communicatively coupled with the ASBs 104 via wired or wireless connections. It should be appreciated that in alternate embodiments, the illumination devices 102 can be configured to emit beacons and the mobile devices can be configured to read the beacons emitted by the illumination devices 102 for localization. The mobile devices can then share their location information with the lighting system (e.g., via a cloud-based connection).

The illumination devices 102 are arranged to provide one or more visible lighting effects 105 which can include a flashing of the one or more LEDs. A flashing of the one or more LEDs can include activating the one or more LEDs at a certain level at regular intervals for a period of time and deactivating or dimming the one or more LEDs a certain amount between the regular intervals when the LEDs are active. It should be appreciated that, when flashing, the LEDs can be active at any a specific level or a plurality of levels. It should also be appreciated that the LEDs can flash at irregular intervals and/or increasing or decreasing lengths of time. The one or more LEDs can also provide a visible lighting effect including one or more changes of color. The color changes can occur at one or more intensity levels. The illumination devices 102 can be controlled by a central controller 112. For example, as described herein the controller 112 can control the illumination devices 102 together or individually based on the position information determined by the ASBs 104 for persons A, B, C, D, and E and their associated mobile devices 106. In example embodiments, the LEDs of the illumination devices 102 can be activated, deactivated, and dimmed by the controller 112. Additionally, the spectral power distribution of the LEDs can be adjusted by the controller 112. Any suitable lighting characteristic can be controlled by controller 112.

Controller 112 includes a network interface 120, a memory 122, and one or more processors 124. Network interface 120 can be embodied as a wireless transceiver or any other device that enables the connected luminaires to communicate wirelessly with each other as well as other devices including mobile devices 106 utilizing the same wireless protocol standard and/or to otherwise monitor network activity and enables the controller 112 to receive data from the ASBs 104. In embodiments, the network interface 120 may use wired communication links. The memory 122 and one or more processors 124 may take any suitable form in the art for controlling, monitoring, and/or otherwise assisting in the operation of illumination devices 102 and performing other functions of controller 112 as described herein.

As shown in FIG. 1A, the beacon receivers 103 are configured to receive beacon signals from mobile devices 106 carried by individuals A, B, C, and D and locate the mobile devices 106. For example, the beacon receivers 103 can be arranged at specific fixed locations within building space 10 and each receiver 103 can store position information indicative of its specific location. This position information can be stored locally at the ASB 104 and/or at memory 122. The beacon signals received from the mobile devices 106 include unique identifying information of that device. The ASBs 104 comprise sensor elements to carry out the functionalities described herein. For example, ASBs 104 can include on-board microphone sensors, multiple-pixel thermopile infrared sensors, ZigBee transceivers, Bluetooth® radio, light sensors, IR receivers, low-resolution image sensors etc. As mentioned above, in embodiments the beacon receivers 103 can also be incorporated within the ASBs 104.

As shown in FIG. 1B, the beacon receivers 103A can also be arranged within the mobile devices carried by individuals A, B, C, and D and the illumination devices 102A can be configured to emit beacon signals including unique location identifying information. The mobile devices can be configured to locate themselves based on the beacon signals received from the illumination devices 102A. The beacon signals received by the mobile devices can include unique identifying information of the illumination device 102A or the position where the illumination device 102A is located. Each mobile device can determine its location and share its location information with the system 100A. FIG. 1B shows system 100A which is otherwise substantially similar to system 100 shown in FIG. 1A. Controller 112A includes a network interface 120A, a memory 122A, and one or more processors 124A configured to operate as discussed above.

As shown in FIGS. 1A and 1B, the systems and methods described herein can be used to provide contact tracing information for persons A, B, C, D, and E in building space 10 and 10A. The embodiments described herein rely on mobile phone-based contact tracing used in parallel with ASBs to monitor social distances within their respective fields of view (FOV). Advantageously, the hybrid systems and methods described herein reduce the possible large proximity estimation errors encountered when using only mobile phone-based contact tracing systems. Additionally, the hybrid systems and methods described herein are able to generate contact tracing information in areas not covered by ASBs alone (e.g., in toilets or far corners). The hybrid systems and methods described herein are also able to track people in densely populated areas whereas ASBs alone have difficulty doing so. For example, ASBs alone cannot accurately track three people moving in the same space when their motion trajectories frequently intersect or one of the persons has reversed his/her direction. The luminaires equipped with contact-tracing data beacon receivers 103 can recognize if a person's phone has reversed its trajectory. Alternatively, the microphone array of the ASBs can detect an angle of arrival and hence based on audio data received of an employee talking on a phone, the lighting system can infer whether he/she has reversed his walking trajectory.

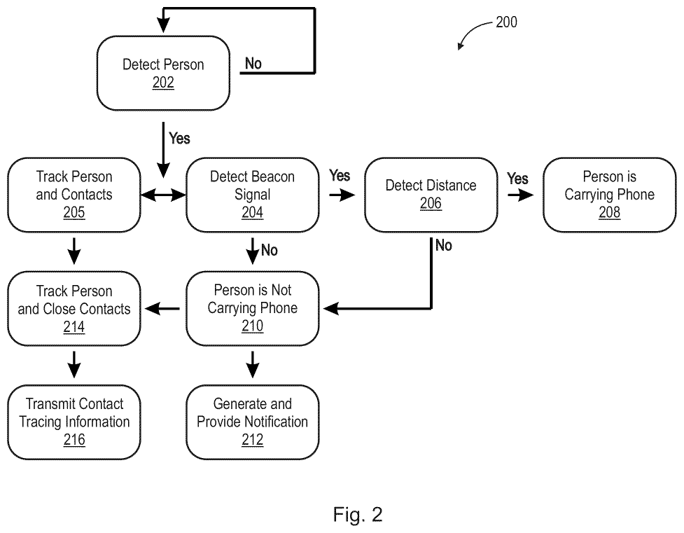

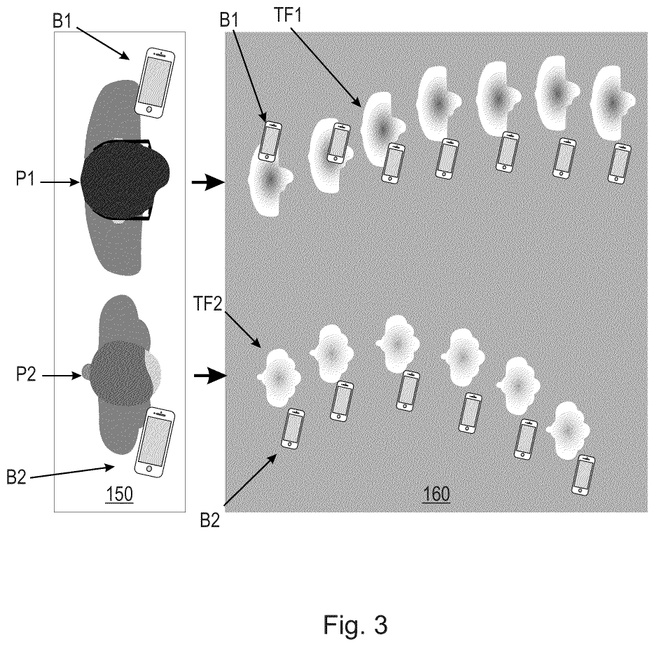

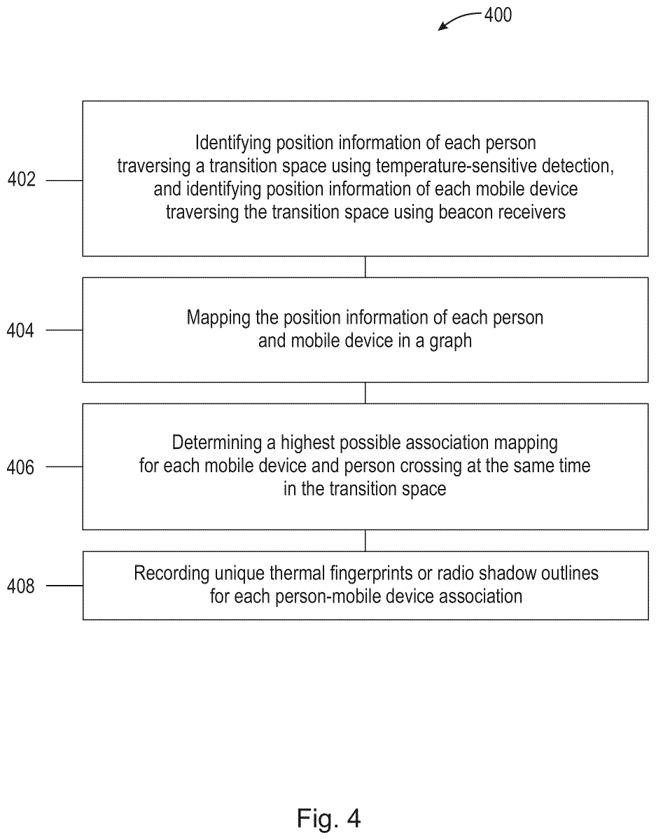

The following should be considered in view of FIGS. 2, 3, and 4. FIG. 2 shows example processes 200 for enforcing contact tracing and complementing mobile phone-based contact tracing. FIGS. 3 and 4 show example depictions and methods of associating two people with two mobile devices in a transition area 150, respectively. At step 202 of FIG. 2, the processes described herein begin with detecting persons A, B, C, D, and E and associating the detected individuals with their mobile devices so that they can be tracked throughout the whole building indoor space 10. As shown in FIG. 2, if no new individual is detected with the ASBs 104, the process repeats the detecting step until a new individual is detected. The new individual can be detected by one or more multi-pixel thermopile infrared sensor arrays in the ASBs or any suitable alternative.

To carry out the associating of people with phones, the detecting step 202 can take place in a transition space 150 where almost everyone passing carries their mobile device. Areas of ingress and egress are particularly suitable since all people will traverse those areas. Example transition spaces include but are not limited to an entrance area to a building or an office space within a building, a staircase, a corridor, an elevator lobby, bathroom exits, or in front of a coffee machine etc. In embodiments, the transition space 150 can be equipped with one or more forward-looking infrared (FLIR) thermal cameras to measure the body temperature of the arriving individuals. FLIR images can be used to record person-specific unique thermal features which can be used later by the much lower resolution thermopile sensing system described herein.

At step 204 of FIG. 2, one or more beacon receivers 103, which are also arranged in the same transition space 150, detect beacon signals transmitted by each mobile device 106 to locate the phones carried by the individuals passing through the transition space 150. Such localization can be carried out using RSSI or any suitable alternative to determine distance estimates. When there is a large number of individuals typically traversing the transition space, embodiments can include a large number of beacon receivers 103 in the transition space to increase the phone-person association accuracy. Since the beacon receivers 103 are positioned in a transition space 150 without intervening walls, the position information of the mobile devices 106 is less prone to estimation errors. In embodiments, the step of detecting the presence of mobile devices 106 can be carried out with thermopile infrared sensors of the ASBs 104. For example, the thermopile sensors of the ASBs can detect the presence of the mobile device 106 in a pocket or in a hand based on a cooler phone blocking IR radiation of the body. This is possible as phones have a distinct shape. In these embodiments, the thermopile sensors include additional pixels to carry out this additional functionality.

At the transition space 150, a person not carrying a phone can be detected due to a mismatch of a BLE-based phone count and a thermopile-based person count. Subsequently, all of the detected mobile devices and people are tracked and labeled accordingly by the beacon receivers 103 and thermopile sensors in the ASBs 104 as they traverse the transition space 150. FIG. 4 shows further steps of an example process 400 of associating two people with two mobile devices in a transition area involving capturing a thermopile image and associating mobile devices with persons as seen.

Referring to FIG. 4, in example embodiments associating two people with two mobile devices begins at step 402 with identifying position information of each person traversing the transition space using temperature-sensitive detection (e.g., thermopile infrared detection). For example, the position information can be recorded as Pₘ(xₙ, yₙ), for the nth position of the mth person-object. Step 402 further includes identifying position information of each mobile device traversing the transition space using the beacon receivers, for example. In embodiments, the position information can be recorded as Bᵢ(xⱼ, yⱼ) for the jth position of the ith object. The previous equations assume m, i={0, . . . , M−1}, n, j={0, . . . , N−1}.

At step 404 in FIG. 4, such position information identified in step 402 can be mapped in one graph as depicted in FIG. 3. When person P1 carries phone B1 and person P2 carries phone B2 through transition space 150, the position information of persons P1 and P2 and phones B1 and B2 identified in step 402 can be mapped onto a single graph 160 as shown in FIG. 3.

At step 406 in FIG. 4, associating two people with two mobile devices proceeds by determining a highest possible association mapping between a phone and an individual crossing at the same time in the transition space 150 using hypothesis tests. In example embodiments, the hypothesis tests are run by a maximum likelihood test algorithm. As shown in FIG. 3, although the lighting system can experience beacon positioning errors, statistically person P1 is closer to phone B1 compared to the time-series distance between person P1 and phone B2. In embodiments, distance can be used to calculate the mapping probability between a phone and people (m, i), assuming a normal distribution of the distance between a person and a phone as shown in the following equation:

$$Prob ( m , i ) = {\prod }_{j} {\prod }_{n} Prob ( {B}_{i} ( {x}_{j} , {y}_{j} ) , {P}_{m} ( {x}_{n} , {y}_{n} ) ) )$$

Using the above equation, a M×M probability matrix can be provided and all probabilities of all possible combinations of (M!) can be calculated as shown below in Table 1.

TABLE 1

| Probability(B, P) | B1 | B2 |
|---|---|---|
| P1 | 0.35 | 0.15 |
| P2 | 0.20 | 0.30 |

Then, the highest probability of all of the probabilities of all possible combinations of (M!) is selected as shown below with the “^{•}” symbol in Table 2:

TABLE 2

| | Probability | |
|---|---|---|
| P(B1, P1)P(B2, P2) | 0.105 | * |
| P(B1, P2)P(B2, P1) | 0.030 | |

As shown in Table 2, the hypothesis test will choose the combination P(B1, P1)P(B2, P2) since its value is higher than the alternative combination P(B1, P2)P(B2, P1). Since only two people are present in the transition area 150 in the example shown in FIG. 3, the number of all possible combinations is equivalent to the factorial of 2 (e.g., 2! which equals 2). However, if there are additional people in different embodiments, then additional phone-person combinations must be analyzed. For example, if there are 5 people and 5 mobile devices present in the transition space 150, the hypothesis test would analyze a total of 5! which equals 125 device-person combinations. In example embodiments, a maximum-likelihood sequence estimation algorithm (like Viterbi algorithm) could effectively reduce the search time if M is greater than 5.

At step 408 in FIG. 4, associating two people with two mobile devices proceeds by recording unique thermal profiles or fingerprints of the detected individuals with the temperature-sensitive sensors. As shown in FIG. 3, since person P1 is taller than person P2, the temperature recorded for person P1 by the ceiling mounted thermopile sensing system is higher than the temperature recorded for person P2. Thus, the different physical properties of the detected individuals (e.g., height) results in different “unique” thermal profiles, for example, thermal fingerprint TF1 for person P1 and thermal fingerprint TF2 for person P2. The thermal profiles can also include additional features such as cluster area and peak temperature. The unique thermal profiles assist with the lighting system tracking different people across the building space. It should be appreciated that the tracking of different people can also be accomplished with other technologies, e.g., 60 GHz Wi-Fi radiofrequency sensing generating a radio shadow outline of the people (via beamsteering of the radio transmission).

After performing the association of people with mobile devices in the transition space as described herein, the mobile devices B1 and B2 are associated with the thermal image profiles of their associated persons P1 and P2, respectively, and their motion patterns throughout the building space. Unlike FLIR cameras, ASBs take low-resolution images, typically 16×16 or 24×32 from the ceiling. Hence, a person is not recognizable from the low-resolution images provided by the thermopile sensors. Despite the coarse thermal images generated by the thermopile sensors, to preserve privacy of the detected individuals, the ASBs can perform the tracking for timeslots of 30 seconds only and all tracking data can be erased after each timeslot.

Additionally, on occasion the track link between a person and their associated mobile device can be lost, for instance, when the person enters a blind spot area without ASB and/or BLE coverage. In these circumstances, upon the person re-entering part of the building space with ASB coverage, another hypothesis test can be initiated by the system. This additional hypothesis test can take into account the known links for other mobile devices and people concurrently in the same space.

Referring back to FIG. 2, after the people and mobile devices are associated, the ASBs monitor the whereabouts of the individuals by proceeding to step 202 again. At step 202, as the people move about the building space one or more ASBs detect a new individual within its field of view (FOV). If an ASB detects a person at step 202, the system can search for new beacon signals via a correspondingly located beacon receiver at step 204. Additionally, at step 205, the system can track the person detected at step 202 and the contacts of the detected person even if the new beacon signals are not yet detected at step 204.

The thermopile sensors and BLE radio signal receivers can be used to detect if a person is carrying their associated mobile device. At step 206, the ASBs can detect whether the person and the mobile device are within a predetermined distance (e.g., <1 m) after new beacon signals are detected at step 204. At step 208, if the person and the mobile device are within the predetermined distance, the system can determine that the person is carrying his/her mobile device. If the location of the mobile device and the location of the person coincide, a new tracking timeslot can be initiated and the previously recorded tracking data can be erased. This ensures that the system does not generate any continuous monitoring of the daily activities of the individuals.

However, if the location of the mobile device and the location of the person do not coincide (e.g., if no device is detected within a predetermined distance at step 204), then the system can determine that the detected individual is not carrying his/her mobile device at step 210. In other words, the ASBs can determine that there is a distance between the person and the mobile device, and the distance is equal to or above a predetermined threshold value (e.g., 1 m) thus, the ASBs can determine that the person is not carrying their associated mobile device. It should be appreciated that the predetermined threshold can be increased or decreased depending on the application. In embodiments, the ASB multi-pixel thermopile sensor array traces and records the location of a device-free person and the location of a device-carrying person close by the illumination device. The BLE radio embedded in the lighting infrastructure can be used to detect and locate the presence of the device of the device-carrying person. While a person can be free of a mobile device, the ASB thermopile sensors can detect that the device-free person is in social distance of another unidentified person. Since the thermopile infrared sensor arrays rely on infrared radiation which cannot penetrate walls, the ASBs can determine with confidence whether the two people that are deemed to be in close proximity to one another are in the same room unlike BLE-based contact tracing smartphone applications which can encounter difficulty in determining whether individuals are in the same room. As mentioned, said ‘close proximity’ may be phrased as ‘within a predetermined proximity’.

Once the system determines that a person is not carrying his/her mobile device, a notification can be generated at step 212 to remind the person to retrieve his/her mobile device. Such a notification can be provided in real-time by the illumination devices 102, for example, by flashing the illumination devices in any suitable manner or changing one or more colors of the illumination devices. The illumination devices can be individually controlled such that only those illumination devices positioned at or close to where the person is detected provide the notification. In embodiments, a plurality of illumination devices surrounding the location of the person can provide the notification. Any suitable combination of LEDs is contemplated. For example, every other illumination device 102 in a circular pattern surrounding the location of the person can be illuminated to provide a flashing pattern. In other examples, every second illumination device in a rectangular pattern surrounding the location of the person can be illuminated to provide a flashing pattern. The notification can indicate a policy violation in example embodiments. Additionally or alternatively, the policy violation can be recorded and a message can be generated and transmitted to an administrator or facility manager to enforce the policy. When the illumination devices provide the notification, the lighting effect can also signal to other area occupants about the rogue employee and can hence provide social pressure to comply with the policies.

Once the system determines that the person is not carrying his/her phone, additionally or alternatively, he/she can be assigned an ID with his/her profile comprising identifying information (phone number, email, etc.) to be non-anonymously tracked by the system. In alternate embodiments, a semi-anonymous profile may be used similar to the one used by the mobile phone-based contact tracing application. In embodiments, the system can query the mobile device that is positioned at least the predetermined distance away from its associated person and request contact tracing identity information of the owner of the phone. Subsequently, the system can relay the respective contact tracing data to other mobile devices detected within the predetermined distance even though the devices are outside of the BLE range of communication.

In embodiments, throughout steps 202 through 208 the system can be operated according to a first privacy mode where the ASBs record the whereabouts of the associated individuals anonymously. For example, once a person is associated with his/her mobile device, the ASBs anonymously track him/her entering the building space and walking to his/her particular workspace using the unique thermal fingerprints discussed herein. However, when the system determines that a person is not carrying his/her mobile device at step 210, the system can be configured to activate a second privacy mode where the ASBs use an ID and/or identifying information obtained from the mobile device to record the whereabouts of the individual not anonymously as discussed above. In embodiments, such a second privacy mode can be activated when the system determines that a person is not carrying his/her mobile device for a predetermined period of time that meets or exceeds a predetermined threshold amount of time. In embodiments, such a second privacy mode can be activated only after the system determines (1) that the person is not carrying his/her mobile device and (2) that the person not carrying his/her mobile device has encountered another person within social distance. Thus, the ID and/or identifying information can be obtained from the person's mobile device only after the person actually encounters another person within social distance. A temporal component can also be included in such embodiments as discussed above. In further embodiments, before activating such a second privacy mode and after determining that the person is not carrying his/her mobile device, the system can be configured to request that the person confirm whether he/she is close to his/her mobile device using a user feedback module. If the person does not confirm that he/she is close to his/her mobile device in response to the request, perhaps within a predetermined amount of time, then the system activates the second privacy mode that is more invasive.

Additionally, at step 214 the system can track the person detected to have forgotten his/her mobile device at step 210 and the contacts of the detected person who are within a predetermined distance where the predetermined distance is within a predetermined threshold value. The people who are detected within the predetermined distance are considered to be in close proximity to the person who forgot his/her mobile device, or within social distance. The predetermined threshold value can be approximately 6 feet or any other suitable distance value depending on the transmission characteristics of the specific infection or disease intended to be targeted in the particular application. In embodiments, the predetermined distance may not be a fixed distance, and may have a temporal component as well. Thus, in embodiments the system can track the person detected to have forgotten his/her mobile device at step 210 and the contacts of the detected person who are within a predetermined distance for at least a predetermined amount of time. For instance, the system can be configured to track contacts only if the contact of the detected person stays within close proximity for at least a predetermined minimum period of time (e.g., 2 minutes). In embodiments, the system can track the person detected to have forgotten his/her mobile device at step 210 and the contacts of the detected person based on current air flow profiles, characteristics of the specific room (e.g., high ceiling versus low ceiling), etc. Thus, in embodiments the system can track the person detected to have forgotten his/her mobile device at step 210 and the contacts of the detected person who are within a predetermined distance based on a property of the air flow surrounding the individuals or the space in which the individuals are situated. These factors can also be combined with a temporal component as described above.

At step 216, the system can transmit tracing data (e.g., a chirp) on behalf of the phone that is separated from its associated person to those contacts who are within the predetermined distance (e.g., within social distance). For example, the system can determine that person A walked away from his/her desk and left his/her phone at his/her desk. As person A continues to walk away from his/her phone, the system tracks the location of person A and determines that person A encounters person B at a water fountain. In example embodiments, an illumination device 102 above or near the water fountain can transmit data such as a BLE chirp on behalf of person A's phone to person B's phone so that person B's phone can appropriately record all received data chirps using a mobile phone-based contact tracing application. In this way, the social contact between person A and person B will not be missed by person B's contact tracing system even though person A was not carrying his/her phone when he/she encountered person B at the water fountain. It should be appreciated that person B need not be associated with his/her phone. In embodiments, as long as person B carries his/her phone, it is sufficient that person A is associated with his/her phone so that the illumination device 102 can transmit data on behalf of person A's phone to person B's phone and receive data on behalf of person A's phone from person B's phone. Person B's phone can run a BLE contact tracing phone application only. The BLE chirp transmitted by the illumination device can include the ID and/or identifying information obtained from person A's phone to identify person A as discussed above. The illumination device above or near the water fountain can also receive a BLE chirp from person B's phone and the illumination device can relay the BLE chirp via a wireless lighting network to another illumination device above or near person A's phone. The illumination device above or near person A's phone can then transmit the BLE chirp to person A's phone and the data of the BLE chirp is recorded. Advantageously, the social contact between person A and person B will not be missed by person A's contact tracing system even though person A was not carrying his/her phone when he/she encountered person B at the water fountain. As described herein, the tracing data from the lighting system provides complementary information to the tracing data natively recorded by the BLE contact tracing phone applications.

In embodiments, the contact tracing information recorded by the IoT system is sent to the phones of all related people, both the people who are deemed to be carrying their mobile phone (persons A, B, C, and D as shown in FIGS. 1A and 1B) as well as the rogue person not carrying their mobile phone (person E as shown in FIGS. 1A and 1B). If person A tests positive for a virus, person A can use the mobile phone-based contact tracing application to upload all of the data chirps transmitted from person A's phone over a period of time, e.g., a previous two week period, to a database. In the database, the chirps from person A's mobile device can be compared with chirps received from other mobile devices. If any of the chirps from person A's mobile device match chirps picked up by another mobile device, a notification be can generated and transmitted to inform the person associated with the other mobile device of a possible exposure to the virus.

In embodiments, the ASBs can be configured in other transition areas within the building space to check to see if the association link between a phone and a person is correct. Thus, association checks can be performed in additional areas after the initial association step is completed as discussed above.

The hybrid systems and methods described herein can also be used in case there are two rogue individuals (person A and person C) moving throughout the building space without mobile phones. In embodiments where person A and person C encounter person B within social distance but person A and person C are not carrying their mobile phones, it is possible that the ASBs of the IoT system attempt to trace person A and person C separately but fail when person A and person C come into such proximity that is beyond the resolution of the ASBs. Hence, after such an encounter person A and person C can no longer be uniquely identified by the luminaire-based contact tracing system. In embodiments, the ASBs are configured to activate a third mode of operation where the thermopile infrared sensors of the ASBs record the full motion trail of person A and person C at a very fast sampling period (e.g., 1 second) until person A and person C are reunited with their respective phones.

As described above, the lighting system-based contact tracing systems and methods provide an advantageous supplement to guarantee sufficient performance in specific yet critical corner cases currently faced by BLE-based phone contact tracing applications.

It should also be understood that, unless clearly indicated to the contrary, in any methods claimed herein that include more than one step or act, the order of the steps or acts of the method is not necessarily limited to the order in which the steps or acts of the method are recited.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for enforcing contact tracing, comprising:
associating, via a processor associated with a plurality of connected sensors, a first person with a first mobile device based at least in part on a first distance between the first person and the first mobile device;
determining, via the processor, that the first person and the first associated mobile device are separated from each other by a second distance; and
notifying, via at least one light effect provided by a first illumination device in communication with the processor, the first person that the first person and the first associated mobile device are separated from each other by the second distance
determining, via the processor, when the first person and the first associated mobile device are separated from each other by the second distance, that a second person having a second mobile device is positioned within a predetermined proximity to the first person; and
receiving, via the first illumination device, contact tracing data on behalf of the first mobile device from the second mobile device.

2. The method of claim 1, wherein the associating step comprises calculating a mapping probability between the first person with the first mobile device and comparing the mapped probability between the first person with the first mobile device with another mapping probability between the first person or the first mobile device with the second person or the second mobile device.

3. The method of claim 1, further comprising the step of tracking the first person through a space by recording a thermal profile or a radio shadow outline of the first person.

4. The method of claim 1, further comprising the step of transmitting, via the first illumination device, contact tracing data on behalf of the first mobile device to the second mobile device.

5. The method of claim 1, further comprising the steps of transmitting, via the first illumination device, the received contact tracing data to a second illumination device that is positioned closer to the first mobile device than the first illumination device, and transmitting, by the second illumination device, the transmitted contact tracing data to the first mobile device.

6. The method of claim 1, wherein the step of determining that the second person is positioned within the predetermined proximity comprises:
determining that the second person is positioned within a third distance from the first person; or
determining that the second person is positioned within the third distance from the first person for at least a predetermined amount of time.

7. The method of claim 1, further comprising the steps of: (i) activating a first mode of operation when it is determined that the first person is carrying the first associated mobile device; and (ii) tracking the first person in the first mode of operation.

8. The method of claim 7, further comprising the steps of: (i) determining that the first person and the first associated mobile device are separated from each other by the second distance for a period of time that meets or exceeds a predetermined threshold; and (ii) initiating a second mode of operation comprising retrieving identifying information of the first person from the first mobile device at least in part in response to determining that the first person and the first associated mobile device are separated from each other by the second distance for the period of time.

9. A system for enforcing contact tracing, comprising:
at least one beacon receiver configured to identify position information for locating a first mobile device;
at least one temperature-sensitive sensor associated with a plurality of connected sensors configured to identify position information of a first person;
a processor associated with the plurality of connected sensors, wherein the processor is configured to associate the first mobile device with the first person based at least in part on a first distance between the first person and the first mobile device, wherein the first distance is based on the position information identified by the at least one beacon receiver and the at least one temperature-sensitive sensor, characterized in that the processor is further configured to determine that the first person and the first associated mobile device are separated from each other by a second distance; and
a first illumination device configured to provide at least one light effect to notify the first person that the first person and the first associated mobile device are separated from each other by the second distance,
wherein the processor is further configured to determine, when the first person and the first associated mobile device are separated from each other by the second distance, that a second person having a second mobile device is positioned within a predetermined proximity to the first person, and the first illumination device is further configured to receive contact tracing data on behalf of the first mobile device from the second mobile device.

10. The system of claim 9, wherein the processor is further configured to track the first person through a space by recording a thermal profile or radio shadow outline for the first person, and the processor is further configured to track the first person according to a first mode of operation prior to determining that the first person and the first associated mobile device are separated from each other by the second distance.

11. The system of claim 10, wherein the processor is further configured to track the first person through the space according to a second mode of operation after it is determined that the first person and the first associated mobile device are separated from each other by the second distance for a period of time that meets or exceeds a predetermined threshold, wherein in the second mode of operation, the plurality of connected sensors are configured to retrieve identifying information of the first person from the first associated mobile device.

12. The system of claim 9, wherein the first illumination device is further configured to transmit contact tracing data on behalf of the first mobile device to the second mobile device, and the first illumination device is further configured to transmit the received contact tracing data to a second illumination device positioned closer to the first mobile device than the first illumination device, and the second illumination device is configured to transmit the received contact tracing data to the first mobile device.

13. A computer program product embodied on at least one non-transitory computer-readable storage medium and configured so as when executed on one or more processors of a lighting system performs the following:
associate a first mobile device with a first person based on position information identified for the first mobile device and the first person;
track the first person throughout a space;
determine when the first person and the first associated mobile device are separated from each other by a first distance;
detect, after determining that the first person and the first associated mobile device are separated from each other by the first distance, that a second person having a second mobile device is positioned within predetermined proximity to the first person;
cause an illumination device to provide at least one light effect to notify the first person that the first person and the first associated mobile device are separated from each other by the first distance; and
cause transmission of contact tracing data between the first and second mobile devices.
